(19) United States
(12) Patent Application Publication
(10) Pub. No.: US 2019/0262175 A1
(43) Pub. Date: Aug. 29, 2019

(54) SYSTEM FOR PERFORMING PHACOEMULSIFICATION

(71) Applicant: FRITZ RUCK OPHTHALMOLOGISCHE SYSTEME GMBH, Eschweiler (DE)

(72) Inventors: Franciscus Theodorus Kerkhoff, Waalre (NL); Manfred Klomp, Hulsberg (NL)

(21) Appl. No.: 16/331,352

(22) PCT Filed: Sep. 1, 2017
(86) PCT No.: PCT/EP2017/071938

§ 371 Date: Mar. 7, 2019

(30) Foreign Application Priority Data
Sep. 14, 2016 (EP) 16188792.2

(51) Int. Cl. A61F 9/007; A61M 3/02; A61M 1/00

(52) U.S. Cl. CPC A61F 9/00745; A61M 3/0216; A61M 1/0064; A61M 3/0258; A61B 2217/007; A61M 2205/3337; A61M 2210/0612; A61B 2217/005; A61M 3/0283

(57) ABSTRACT

A system, including a handpiece with a tool formed by a hollow needle and a lateral surface surrounding the hollow needle, where the hollow needle forms a first channel and a second channel is formed between the hollow needle and the lateral surface, an irrigation device, an aspiration device, a manifold device and a control device. In a first operating mode, the manifold device connects the second channel and the irrigation device for the exchange of fluids and connects the first channel and the aspiration device for the exchange of fluids. In a second operating mode, the manifold device connects the first channel and the irrigation device for the exchange of fluids. The pressure at which the irrigation device delivers fluid to the manifold device is controllable linearly in the second operating mode by the control device.

The invention relates to a system for performing a phacoemulsification, comprising a handpiece with a tool formed by a hollow needle and a lateral surface surrounding the hollow needle, wherein the hollow needle forms a first channel and a second channel with an annular cross-section is formed between the hollow needle and the lateral surface, an irrigation device configured for delivering a fluid at a pressure, an aspiration device configured for aspirating the fluid, a manifold device, wherein the manifold device is connected to the first channel, the second channel, the irrigation device and the aspiration device for the exchange of fluids, and a control device which is connected to the irrigation device, the aspiration device and the manifold device for the exchange of information, wherein the manifold device in a first operating mode connects the second channel and the irrigation device for the exchange of fluids and connects the first channel and the aspiration device for the exchange of fluids and wherein the manifold device in a second operating mode connects the first channel and the irrigation device for the exchange of fluids.

In order to perform a phacoemulsification on the eye, the eye is first opened by an incision in the cornea. In doing so, the incision must be configured so large that a new lens can be introduced into the eye in a rolled-up condition. Through the incision, a capsular sac of the eye, in which the lens of the eye is seated, is opened circularly by means of a needle. In order to detach the lens from the capsular sac, a very thin cannula is subsequently inserted into the incision and pushed between the capsular sac and the lens. The cannula is connected to a syringe by means of which a surgeon is able to inject fluid between the lens and the capsular sac for detaching the lens. Excess fluid flows out of the eye through the incision, passing a shaft of the cannula, wherein a gap between the cannula and the incision is very large due to the thin cannula and because of the size of the incision. Subsequently, a tool attached to a handpiece is inserted through the incision into the eye for performing the phacoemulsification. The tool is formed by a hollow needle and a lateral surface surrounding the hollow needle, wherein the hollow needle forms a first channel and a second channel with an annular cross-section is formed between the hollow needle and the lateral surface. The hollow needle is induced to vibrate in the ultrasonic range, whereby the lens can be smashed into small parts, with split-off parts of the lens being sucked from the eye through the first channel by means of the aspiration device. During the operation, irrigation fluid is constantly supplied to the eye via the second channel in order to keep an internal pressure within the eye as constant as possible. A pressure of the irrigation fluid supplied to the eye is controlled in a stepwise manner using the irrigation device and a control device. Such a system for controlling the pressure is known, for example, from the patent application EP 2 674 176 A1. The regulation of the internal pressure is of utmost importance, since the eye may sustain irreparable damage if the internal pressure is too low and also if the internal pressure is too high. So as to avoid that other tissue gets aspirated through the first channel during the aspiration of the fragmented lens, temporary back-flushing through the first channel is possible, thereby expelling tissue which has been sucked in by mistake.

When the lens is detached from the capsular sac by means of the cannula and the syringe, it has proved to be disadvantageous that the surgeon can control the amount of fluid and thus a fluid pressure at which the fluid is delivered into the eye only poorly with the syringe because of an internal friction of the syringe. As already described, an excessive internal pressure within the eye has to be avoided at all costs. Furthermore, it has proved to be disadvantageous that, due to the size of the gap between the cannula and the incision, an iris may be partly flushed out of the eye due to the fluid flowing out of the eye, passing the shaft of the cannula. This occurs especially in individuals who take medication similar to alpha-blockers, since such medication causes slackening of the iris.

It is the object of the present invention to provide a system for performing a phacoemulsification which can be used for the implementation of further procedures on the eye of a patient.

According to the invention, the object is achieved in that the pressure at which the irrigation device delivers fluid to the manifold device is controllable linearly in the second operating mode by means of the control device.

Due to the design of the system for performing a phacoemulsification which is according to the invention, other procedures may also be performed on the eyes of a patient by using the system. As a result of the linearly controllable pressure at which the irrigation device delivers fluid to the manifold device in the second operating mode, the lens can also be detached from the capsular sac prior to the implementation of the phacoemulsification by means of the system according to the invention. Thus, the use of the cannula and the syringe for detaching the lens from the capsular sac may be omitted, whereby switching of surgical devices is avoided and the surgical procedure can be accomplished faster. Moreover, when the system according to the invention is used, the advantage is obtained that the tool, which is introduced into the eye, has a large diameter on the shaft. As a result, the incision in the cornea which has been defined essentially in its size is filled in better, whereby only a small gap is created between the tool and the incision and the iris is prevented from being flushed out.

Suitably, the irrigation device is formed by a container which can be charged with an overpressure by gas introduced into the container by means of a pump controlled by the control device, whereby the pressure at which the irrigation device delivers fluid to the manifold device is controlled. Advantageously, the control device comprises a foot switch formed by a foot pedal, wherein the control device in the first operating mode and/or second operating mode controls the overpressure and thus the pressure at which the irrigation device delivers fluid to the manifold device in proportion to an angular position of the foot pedal. As a result, the system according to the invention is very easy to handle for a surgeon, and the pressure can be proportioned very well by the surgeon.

In a further embodiment variant, the irrigation device is formed by a container which is displaceable in the vertical direction by means of a lifting device controlled by the control device, whereby the pressure at which fluid is delivered from the container to the manifold device is altered. In this context, it is also advantageous if the control device has a foot switch formed by a foot pedal, wherein the control device in the first operating mode and/or second operating mode controls a position of the container in the vertical direction in proportion to the angular position of the foot pedal.

Suitably, the control device furthermore comprises a switch which is attached either to the foot switch or to the handpiece and is configured for switching between operating modes. As a result, the advantage is obtained that the surgeon can switch back and forth between the operating modes without being forced to put down the handpiece.

The aspiration device is preferably formed by a cartridge charged with a vacuum or by a peristaltic pump adapted for sucking off the fluid, wherein a negative pressure at which fluid is sucked off can be controlled via the control device.

Further advantageous embodiments of the system according to the invention will be explained in further detail below with reference to the figures.

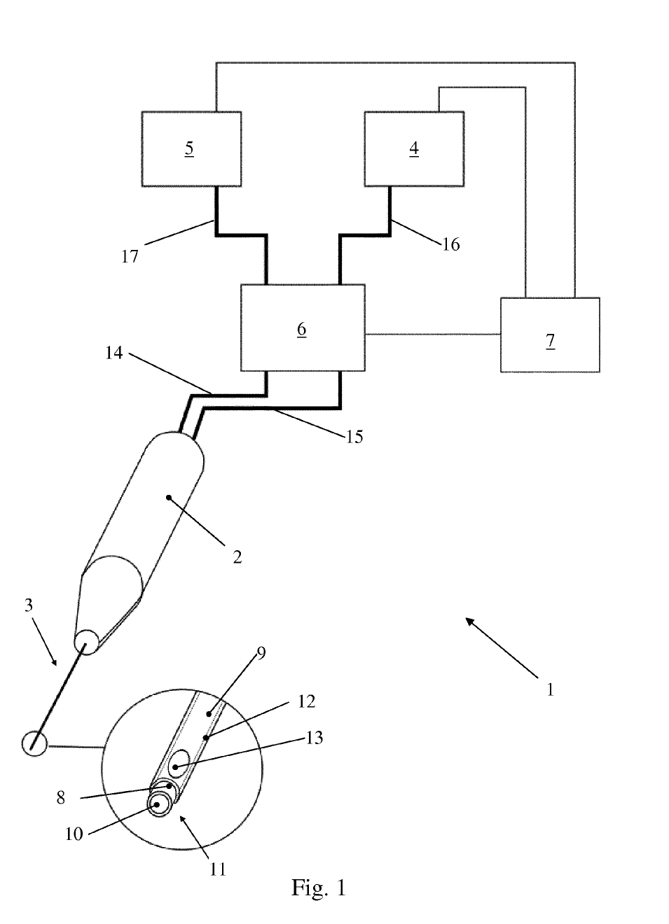

FIG. 1 shows an embodiment variant of a system according to the invention in a schematic view.

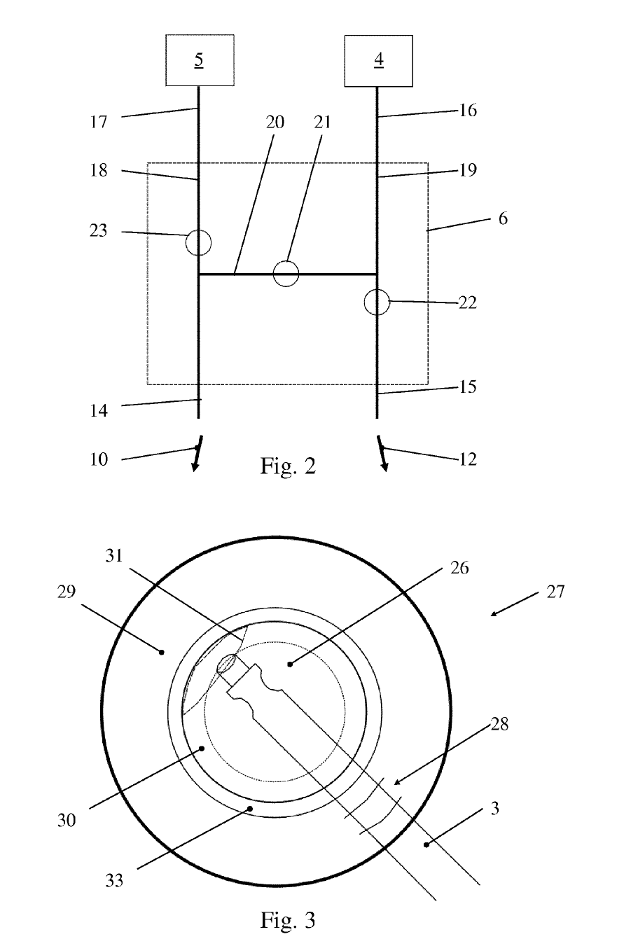

FIG. 2 shows an embodiment variant of a manifold device of the system according to the invention as per FIG. 1 in a schematic view.

FIGS. 3 to 5 show an embodiment variant of a tool of the system according to the invention as per FIG. 1 in a schematic view during a procedure on the eye of a patient with varying progress of the operation.

FIG. 1 shows an embodiment variant of a system 1 according to the invention for performing a phacoemulsification in a schematic view. The system 1 according to the invention comprises a handpiece 2 with a tool 3, an irrigation device 4, an aspiration device 5, a manifold device 6 and a control device 7. The tool 3 is formed by a hollow needle 8 and a lateral surface 9 surrounding the hollow needle 8. The hollow needle 8 forms a first channel 10, which leads to an open end 11 of the hollow needle 8, and is advantageously formed of stainless steel. The lateral surface 9 is advantageously formed by a pipe made of metal or a synthetic material or by a plastic tube, the pipe or tube, respectively, being sealingly closed off at the hollow needle 8 in the region of the open end 11. Between the hollow needle 8 and the lateral surface 9, a second channel 12 having an annular cross-section is formed, which ends in two opposite openings 13. For the exchange of fluids, the manifold device 6 is connected by means of a first line 14 to the first channel 10 of the tool 3, by means of a second line 15 to the second channel 12 of the tool 3, by means of a third line 16 to the irrigation device 4 and by means of a fourth line 17 to the aspiration device 5. For the exchange of information, the control device 7 is connected to the irrigation device 4, the aspiration device 5 and the manifold device 6 and is advantageously formed by a microcontroller or a computer.

The irrigation device 4 is formed by a container which contains a fluid, in particular an irrigation fluid, and by a pump which is designed for supplying a gaseous medium, in particular air, into the container for generating an overpressure in the container. The aspiration device 5 is formed by a peristaltic pump configured for aspirating the fluid. Both the gas supply into the container of the irrigation device 4 and a negative pressure at which fluid is aspirated through the peristaltic pump are controlled by the control device 7. Advantageously, the control device 7 for controlling the gas supply into the container and thus for controlling a pressure at which fluid is discharged from the container comprises a foot switch formed by a foot pedal, wherein the pressure at which fluid is discharged from the container is in proportion to an angular position of the foot pedal.

FIG. 2 shows an embodiment variant of a manifold device 6 of the system 1 according to the invention as per FIG. 1 in a schematic view. The manifold device 6 comprises an aspiration line 18 connecting the aspiration device 5 and the first channel 10, an irrigation line 19 connecting the irrigation device 4 and the second channel 12, a connecting line 20 connecting the aspiration line 18 and the irrigation line 19 and three valves. A first valve 21 is formed in the connecting line 20. A second valve 22 is formed between a region in which the connecting line 20 runs into the irrigation line 19 and the second line 15, which second line 15 runs into the second channel 12. A third valve 23 is formed between a region in which the connecting line 20 runs into the aspiration line 18 and the fourth line 17, which fourth line 17 ends in the aspiration device 5. In FIG. 2, the first channel 10 and the second channel 12 are stylized by respective arrows 10 and 12. The valves of the manifold device 6 are activated by the control device 7.

In a further embodiment variant, the manifold device 6 comprises a bypass line connecting to the irrigation line 19 between the second valve 22 and the second line 15, which runs into the second channel 12, and running into the aspiration line 18 between the third valve 23 and the fourth line 17. A fourth valve is formed in the bypass line.

FIGS. 3 to 5 show an embodiment variant of a tool 3 of the system 1 according to the invention as per FIG. 1 in a schematic view during a procedure on the eye 27 of a patient with varying progress of the operation. The eye 27 of the patient has already been opened by an incision 28 in a cornea 29 of the eye 27, and a capsular sac 30 of the eye 27 has already been opened in an essentially circular manner by means of a needle.

Subsequently, an application of the system 1 according to the invention for detaching a lens 26 from the capsular sac 30 of an eye 27 and for performing a subsequent phacoemulsification is described in further detail. The system 1 according to the invention can be operated in two operating modes, wherein, in the first operating mode, the first valve 21 is closed and the second valve 22 and the third valve 23 of the manifold device 6 are opened. As a result, the irrigation device 4 is connected to the second channel 12 for the exchange of fluids, and the aspiration device 5 is connected to the first channel 10 for the exchange of fluids. Furthermore, in the first operating mode, the tool 3 is made to vibrate mechanically with the aid of drive means (not illustrated) formed within the handpiece 2. In a second operating mode, the second valve 22 and the third valve 23 are closed and the first valve 21 is opened. As a result, the irrigation device 4 is connected to the first channel 10 for the exchange of fluids.

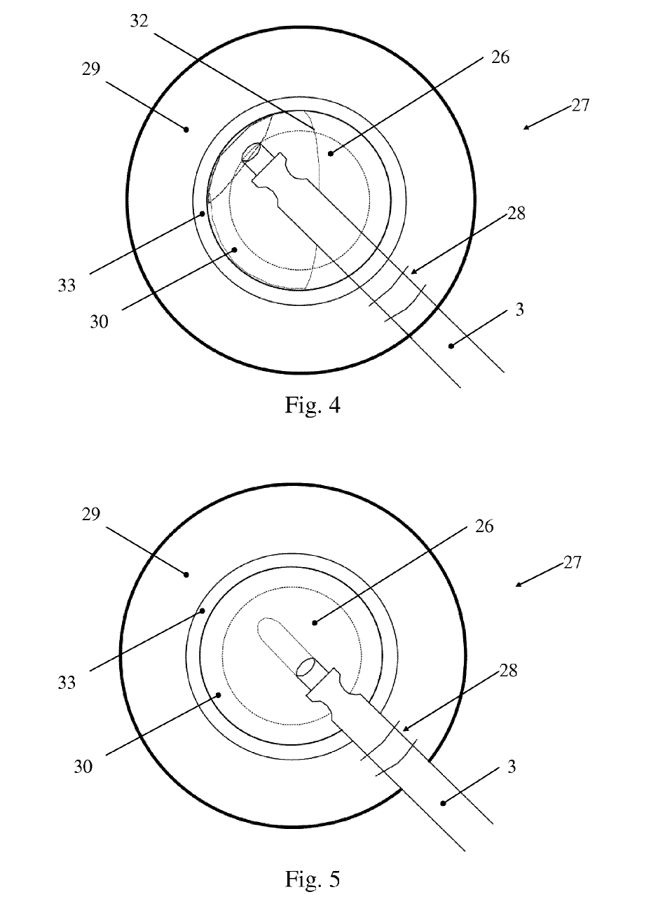

For detaching the lens 26 from the capsular sac 30, the surgeon adjusts the second operating mode on the control device 7. For this purpose, the control device 7 advantageously comprises a switch, which is formed, for example, on the handpiece 2 or on the foot pedal. To detach the lens 26, the hollow needle 8 is pushed between the lens 26 and the capsular sac 30. Via the foot pedal of the control device 7, the surgeon can now control a pressure at which fluid is discharged from the container of the irrigation device 4 and, consequently, an outlet pressure at which fluid leaves the tool 3 through the first channel 10, with the control device 7 controlling the pressure in such a way that it will be proportional to an angular position of the foot pedal 7 and an outlet pressure at the first channel 10 of about 120 mm WaterColumn will not be exceeded. The fluid is pushed between the lens 26 and the capsular sac 30 due to the pressure and spreads therebetween, thereby separating the lens 26 from the capsular sac 30. In FIG. 3 and FIG. 4, fluid fronts 31 and 32 are depicted with varying progress of the expansion of the fluid within the eye 27. If the lens 26 is detached, the lens can be rotated in the capsular sac 30. During the injection of fluid into the eye 27 via the first channel 10, excess fluid drains out of the eye 27 through a gap between a shaft of the tool 3 and the incision 28, wherein, due to the thickness of the tool 3, the gap is so small that an iris 33 of the eye 27 is prevented from being flushed out of the eye 27. If the manifold device 6 has a bypass line with a fourth valve, the excess fluid can also be sucked out of the eye 27 through the second channel 12 via the aspiration device 5 by opening the fourth valve.

Subsequently, the surgeon switches to the first operating mode by means of the switch of the control device 7 in order to perform the phacoemulsification. In the first operating mode, fluid is delivered into the eye 27 through the second channel 12, and fluid is sucked out of the eye 27 via the first channel 10. Advantageously, in the first operating mode, a pressure at which fluid is discharged from the irrigation device 4 is no longer regulated linearly, but in a gradual manner. As soon as the foot pedal is actuated by the surgeon ever so slightly, the pressure at which fluid is discharged from the irrigation device 4 is adjusted to a fixed preset value. The pressure at which fluid is discharged from the irrigation device 4 in the first operating mode is set and adjusted via a separate adjusting device of the control device 7. As a result, the advantage is obtained that the surgeon is able to concentrate on the surgical procedure and does not always have to adjust the pressure via the angular position of the foot pedal. During the phacoemulsification, the lens 26 is smashed according to FIG. 5 into individual parts which are sucked off via the first channel 10.

In this connection, it should also be mentioned that the manifold device may also be formed by a simple directional valve, in particular a 4-port/2-way valve.

1.-6. (canceled)

7. A system for performing a phacoemulsification, comprising
a handpiece with a tool formed by a hollow needle and a lateral surface surrounding the hollow needle, wherein the hollow needle forms a first channel and a second channel with an annular cross-section is formed between the hollow needle and the lateral surface,
an irrigation device configured for delivering the fluid at a pressure, an aspiration device configured for aspirating the fluid, a manifold device, wherein the manifold device is connected to the first channel, the second channel, the irrigation device and the aspiration device for the exchange of fluids, and a control device which is connected to the irrigation device, the aspiration device and the manifold device for the exchange of information,
wherein the manifold device in a first operating mode connects the second channel and the irrigation device for the exchange of fluids and connects the first channel and the aspiration device for the exchange of fluids and wherein the manifold device in a second operating mode connects the first channel and the irrigation device for the exchange of fluids, and wherein the pressure at which the irrigation device delivers fluid to the manifold device is controllable linearly in the second operating mode by the control device.

8. The system according to claim 7, wherein the manifold device comprises an aspiration line connecting the aspiration device and the first channel of the handpiece, an irrigation line connecting the irrigation device and the second channel of the handpiece, a connecting line connecting the aspiration line and the irrigation line and three valves, wherein a first valve is formed in the connecting line, a second valve is formed in the irrigation line between a region in which the connecting line runs into the irrigation line and the second channel, and a third valve is formed in the aspiration line between the aspiration device and a region in which the connecting line runs into the aspiration line, and wherein, in the first operating mode, the second valve and the third valve are opened and the first valve is closed and, in the second operating mode, the second valve and the third valve are closed and the first valve is opened.

9. A system according to claim 8, wherein the manifold device comprises a bypass line connecting to the irrigation line between the second valve and the second channel and running into the aspiration line between the third valve and the aspiration device, with a fourth valve being formed in the bypass line, which fourth valve is closed in the first operating mode and open in the second operating mode.

10. The system according to claim 8, wherein the control device comprises a foot switch for controlling the pressure and comprises a switch operable by hand or foot for switching between the operating modes.

11. The system according to claim 8, wherein the irrigation device is formed by a container which can be charged with an overpressure by a pump controlled by the control device, and/or that the irrigation device is formed by a container which is displaceable in the vertical direction by a lifting device controlled by the control device.

12. The system according to claim 7, wherein the aspiration device is formed by a cartridge charged with a vacuum or by a peristaltic pump, which peristaltic pump is adapted for aspirating fluid.
